(19) United States
(12) Patent Application Publication
(10) Pub. No.: US 2011/0268816 A1
(43) Pub. Date: Nov. 3, 2011

(54) APPARATUSES AND SYSTEMS TO PROCESS A FLUID, AND METHODS FOR USING THE SAME

(76) Inventor: John Michael DeBoer, Spring, TX (US)

(21) Appl. No.: 12/771,986

(22) Filed: Apr. 30, 2010

(51) Int. Cl. A61K 33/00; B64B 1/40; B64F 1/04

(52) U.S. Cl. 424/613; 244/63; 244/31

(57) ABSTRACT

A method for oxygenating a fluid, the method including launching a sub-orbital lower-level apparatus to a predetermined altitude greater than 20,000 feet above sea level. The method also includes, at the predetermined altitude, actuating a fluid capture mechanism sealingly configured to collect and store an atmospheric fluid. In addition, returning the a sub-orbital lower-level apparatus to an altitude less than 20,000 feet, and introducing at least a portion of the atmospheric fluid to an oxygenation process to mix the captured atmospheric fluid with another fluid.

### BACKGROUND OF DISCLOSURE

1. Field of the Disclosure

Embodiments disclosed herein relate to apparatuses and systems for processing a fluid, and methods for using the same. Other embodiments relate to high altitude acquisition and capture of a first fluid used for mixing with a second fluid. Specific embodiments relate to an above-surface apparatus configured to overcome resistive forces in order to reach a predetermined altitude.

2. Background Art

The atom is the primary basic unit of matter in the universe, and generally consists of a dense, central nucleus surrounded by a cloud of negatively charged electrons. The atomic nucleus 1570 contains a mix of positively charged protons and electrically neutral neutrons, with electrons of an atom bound to the nucleus by electromagnetic force. Similar forces allow groups of atoms to bind to each other form a molecule. An atom that has an equal number of protons and electrons is considered electrically neutral, otherwise it has a positive or negative charge and is called an ion. An atom is classified according to the number of protons and neutrons in its nucleus: the number of protons determines the chemical element, and the number of neutrons determine the isotope of that chemical element.

Each chemical element has at least one isotope with unstable nuclei that may undergo radioactive decay, which may result in a transmutation that changes the number of protons or neutrons in a nucleus. Electrons that are bound to atoms possess a set of stable energy levels (i.e., orbitals) that undergo transitions by absorbing or emitting photons that match the energy differences between the levels. The electrons determine the chemical properties of an element, and strongly influence an atom's magnetic properties.

Modern instruments have been used to show that isotopes have different masses. One particular element of significant interest, including all of the element's isotopes, is oxygen. Oxygen will usually exist in the three common forms (e.g., gas, solid, liquid), and may exist naturally, or may be produced synthetically. For example, oxygen may be produced by fractional distillation of liquefied air, use of zeolites to remove carbon dioxide and nitrogen from air (e.g., PSA), electrolysis of water, as well as other processes.

Triplet oxygen is the ground state of the O₂ molecule (i.e., oxygen bonded with oxygen). The electron configuration of the molecule has two unpaired electrons occupying two degenerate molecular orbitals. In normal triplet form, O₂ molecules are paramagnetic—they form a magnet in the presence of a magnetic field—because of the spin magnetic moments of the unpaired electrons in the molecule, and the negative exchange energy between neighboring O₂ molecules. For example, it has been shown that liquid oxygen is attracted to a magnet to a sufficient extent that, in laboratory demonstrations, a bridge of liquid oxygen may be supported against its own weight between the poles of a powerful magnet.

Singlet oxygen, a name given to several higher-energy species of molecular O₂ in which all the electron spins are paired, is much more reactive towards common organic molecules. In nature, singlet oxygen is commonly formed from water during photosynthesis, using the energy of sunlight. It is also produced in the troposphere by the photolysis of ozone by light of short wavelength.

Naturally occurring oxygen is composed of three stable isotopes, ¹⁶O, ¹⁷O, and ¹⁸O, with ¹⁶O being the most abundant. Fourteen radioisotopes have been characterized, the most stable being ¹⁵O with a half-life of 122.24 seconds (s) and ¹⁴O with a half-life of 70.606 s. All of the remaining radioactive isotopes have half-lives that are less than 27 s and the majority of these have half-lives that are less than 83 milliseconds. The most common decay mode of the isotopes lighter than ¹⁶O is β⁺ decay to yield nitrogen, and the most common mode for the isotopes heavier than ¹⁸O is beta decay to yield fluorine.

The effects of gravity on concentrations of oxygen is apparent by the presence, or lack thereof, in planetary atmospheres. The unusually high concentration of oxygen gas on Earth is the result of the oxygen cycle. This biogeochemical cycle describes the movement of oxygen within and between its three main reservoirs on Earth: the atmosphere, the biosphere, and the lithosphere. The main driving factor of the oxygen cycle is photosynthesis, which is responsible for modern Earth's atmosphere. Photosynthesis releases oxygen into the atmosphere, while respiration and decay remove it from the atmosphere.

A simplified overall formula for photosynthesis is:

6 CO₂+6 H₂O+photons(energy)→C₆H₁₂O₆+6 O₂,

or simply carbon dioxide+water+sunlight→glucose+dioxygen.

The beneficial uses of oxygen are nearly limitless. Through experiments and research, the benefits from an oxygenated fluid become more and more known and understood. Previously it was known that numerous processes advantageously employ oxygenated fluids. For example, an oxygenated fluid may be advantageously used for at least one or more of the following: human consumption; enhanced aerobic activity; therapeutic/restorative needs for the human body, bioremediation of contaminated (e.g., with petroleum products) or oxygen-depleted bodies of water; wastewater treatment; rehabilitation of atrophying lakes; biological oxygen demand (BOD) reduction techniques; water aquaculture (e.g., fish farming, shrimp farming, etc.); hydroponic agriculture; odor suppression; oxygenated saline solutions; fermentation; disinfection, and more.

In addition to supporting life on Earth, oxygen has a known history of “recreational” use, such as in oxygen bars and in sports. Available studies support a performance boost from oxygenated mixtures, especially if ingestion or inhalation occurs during actual aerobic exercise. “Therapeutic” use, may involve the oxygenation of the body (or various parts thereof) by treatment with a fluid that contains oxygen, and is conveyed via a usable medium, such as the fluid.

Suitable human therapeutic use in which an oxygenated fluid may be advantageously employed include, for example, one or more of the following: processes for increasing the oxygen content of blood and tissues; oxygenation of wounds to increase the rate of healing and to reduce infections; oxygenated organ transplant storage media; tumor oxygenation for radiation therapy and chemotherapy; treatment for carbon monoxide poisoning; contact lens treating solutions; oxygenation of blood cells; hydration of cells and tissues; transportation of nutrients throughout the body; detoxification and elimination of wastes; immune system activation and support; slowing the aging process; weight loss by promoting fat burning; fatigue prevention, and more.

Fatigue (e.g., physical tiredness) is known to be the consequence of the anoxia (hypoxia), otherwise described as oxygen insufficiency. Normally, human blood is saturated with oxygen through respiration. In the case of physical exertion, the saturation of the blood with oxygen by respiration may be insufficient because of the limited capacity of the lungs and/or the low oxygen content of the air. For example, a professional athlete may be seen on television going off a field between plays to wear oxygen masks in order to get a “boost” in performance, which may include a placebo or psychological boost. It is also known to supply oxygen through ingestion because it is more effective than respiration of air.

To overcome fatigue, one may consume a fluid or beverage that has a refreshing effect and restoring capacity. Beneficial beverages like these may include an oxygenated fluid, such as, for example, oxygenated water. The consumption of fluids that previously underwent an oxygenation process, and the results thereof, has been studied in clinical tests, with subsequent and accompanying published reports. The effect of consumption of an oxygenated fluid was subjected to long-lasting, widely extended examinations by numerous official bodies, including the National Institute for Physical Education and Sports Hygiene of Hungary. These and other tests confirm the desirability for a human to consume an oxygenated beverage, with even greater benefit for consumption while performing physical work.

Results show the use of the oxygenated fluid may restore the human body after long-lasting and exhausting physical work, such that consumption may strongly benefit sportsmen and women when the need for rapid restoration, regeneration, or overcoming fatigue is important.

Oxygenated water may be water that is infused (e.g., mixed, enriched, enhanced, etc.) with oxygen. People who consume and ingest oxygenated water will get more oxygen into their bloodstream, which may provide at least one or more of the benefits previously described, and more. The human brain makes billions of electrical decisions per second, as rapidly as some of the world's fastest computers. Oxygen is essential to proper neurological activity. Deprived from oxygen, the brain can cease to function in less than 6-7 minutes.

Simply stated, the two greatest needs of the human body are oxygen and then water. Without either of these two elements, the body will die. In a number of exertions/circumstances, it is common for a human to slightly deprive them self of both. Oxygen is needed in the body in order for energy to be released from the food molecules. This is an aerobic process called aerobic respiration, which is the best way to produce energy in the body.

When there is not enough oxygen present in the body, anaerobic respiration occurs. Anaerobic respiration results in only a partial breakdown of food molecules in order to continue producing energy. As a result lactic acid is produced as well. Once the lactic acid reaches certain sections of the body's muscles and blood, the acid causes a human to feel fatigued and tired. This is exacerbated by the stopping of movement, because the body then needs even more oxygen to break down the lactic acid, which leads to, for example, heavy breathing (i.e., panting) after exercising.

Getting more oxygen into the body will advantageously provide more energy, and will beneficially allow more activity for a longer period of time without feeling fatigued. More oxygen in the blood means that aerobic respiration will take place, which means more energy in the body.

Medical research has also shown that oxygen-deprived cells within a body are more likely to become cancerous. Therefore, cells that receive enough oxygen is important for the prevention of cancer. Since oxygenated water provides more oxygen to the cells, then oxygenated water helps keep cells free from cancer.

Oxygen in the body also helps promote the replication of good bacteria in the body. These good bacteria help keep the human body safe from bad bacteria that may cause harm and sickness. As one example, pathogens (i.e., micro-organisms that cause disease) are almost never found in oxygen rich environments.

There are a number of energy drinks marketed today as being helpful or beneficial to the human body, but these drinks have little to no nutritional value, and most of them do not actually provide extra energy. In stark contrast, an oxygenated water beverage is better for a body because of the ingredients in each drink, namely only what the body needs—oxygen and water.

However, with energy drinks the body is given a lot of other ingredients that actually draw away from the desired effect. For example, there is a lot of sugar in almost all energy drinks. This sugar will cause a quick burst of energy, but that burst will only last for a short while. Then the person is left feeling tired when the initial sugar rush passes. This is commonly referred to as “crashing.”

Also, all the other ingredients in the energy drink have to go through the digestive process. Any time there is anything in the body that is being digested; energy has to be taken from the rest of the body in order for the digestive process to take place. So the energy that is needed to power digestion, coupled with the sugar crash, actually leaves the person (or athlete) with less energy than the person had before the drink.

From the time oxygenated water enters the mouth, oxygen begins to be absorbed into the body (into the bloodstream). This absorption continues all the way through the digestive system. Since there is nothing but water and oxygen, the body does not need to draw any energy from the body in order to process the drink.

If a human body becomes dehydrated or the body becomes an anaerobic (oxygen deficient) environment, a number of undesirable yet preventable events may take place. For example, waste products and toxins may no longer be easily flushed out by the body. The immune system stops working properly because communication between the body's 100 trillion cells and 100 billion brain neurons becomes atrophied or suffocated by insufficient oxygen and gradually increasing toxic accumulations.

Along with helping to maintain a healthy immune system, oxygenated water may help detoxify and eliminate harmful substances and restore good communication between body cells. Healthy levels of oxygen also enable human bodies to burn fat instead of glucose, thus preventing the build up of lactic acid in the muscles.

For over 50 years, health care professionals have agreed that oxygen plays an important role in sustaining a healthy body. Without a dependable supply of oxygen, the cells in human bodies cannot function properly. Nutrients, occurring in human diets, such as proteins, carbohydrates, and fats, must have oxygen present to convert their potential energy into usable energy.

The oxygenated fluid may be bottled with oxygen in a concentration sufficient to provide significant physical energy boosts for the athlete while reducing pulse rates, such as by 5-15 beats per minute; oxygenated fluid may provide an increase in mental acuity and clarity for; oxygenated fluids may provide a healthier “upper” than other sugary drinks, coffee, etc.

An oxygenated fluid is further important for consumption because the air in cities is somewhat of an oxygen-deficient environment. Some severe urban polluted areas may have breathable oxygen levels of less than 20%. In addition to the benefits mentioned, there are also physiological effects are occurring after the use of oxygenated beverages. Several companies have begun licensing or selling their oxygenation technologies worldwide to aloe, herbal, homeopathic, and cosmetic manufacturing, where the introduction of purified oxygen in these products may be revolutionary to say the least.

For example, consumption of an oxygen enriched beverage has a favorable effect on well-being and physical performance, for it provides oxygen to the bloodstream through the stomach lining or intestinal wall. In one case, eight test subjects of various ages and differing sex had their blood oxygen contents and their pulse rates determined. Each subject then drank between ½ and ¾ liters of highly oxygenated water. A short period after ingestion of the enriched water, evidence of a pulmonary function bypass was observed through an average blood oxygen level increase of about 30%, and the effect of a concomitant cardiac relief was observed through an average pulse rate reduction of about 10%. Further, the added oxygen tends to reduce the tartness of any carbonation and does not impart any taste to the resulting liquid. Regardless of the use to which oxygenated liquids are put, means for achieving increased levels of dissolved oxygen in a liquid efficiently is desirable, as are means for doing so at high rates of production.

Zeta potential is a term commonly used in colloidal chemistry. When tiny mineral or organic particles are suspended in a fluid, Zeta potential maintains the dispersion or discreteness of the particles in suspension. The higher the Zeta potential the better the dispersion of particles in suspension.

Without zeta potential, life could not exist. The high Zeta potential or negative electrical charge on particles entering the bloodstream may help to increase the dispersion or discreteness of blood cells by helping to enhance the electrical charge on blood colloids which include blood cells. When blood cells are free flowing, they expose maximum surface area to the blood and are therefore able to hold and transport more oxygen and other nutrients throughout the body.

In the heart, medical science knows that that as the heart valves open and close and as blood rushes through these heart valves, the blood spins in a vortexial fashion, creating negative surface charges on the blood cells as they leave the heart. These negative charges offset a wide range of free radicals which may be entering the body through air, water or foods. Without this constant replenishment of the Zeta Potential on our blood, all of us would be dead long before we began reading this section of the website.

Smaller colloids or silicates result in a stronger Zeta Potential. If the colloids or silicates in the “seed” or concentrate get too large, surface area decreases, Zeta Potential suffers and random collisions cause larger growth. This process is repeated until the system is destroyed.

While oxygenated beverages are known, not all oxygen is the same oxygen. For example, oxygen comes in a number of different isotopal forms. The isotopes may have different effect on the human physiology and psyche when consumed. Moreover, modern science has begun to understand that a comparison of the same chemical element may be substantially different at the subatomic level. These differences may also affect humans differently when the chemical is consumed.

Though the word atom originally denoted a particle that cannot be cut into smaller particles, modern science has made it known that the atom is actually composed of various subatomic particles. As previously mentioned, the constituent particles of an atom are the electron, the proton, and the neutron. In the modern model of physics, both protons and neutrons are composed of elementary particles called quarks. The quark belongs to the fermion group of particles, and is one of the two basic constituents of matter—the other being the lepton, of which the electron is an example.

There are six types of quarks, each having a fractional electric charge of either +⅔ or −⅓. Protons are composed of two up quarks and one down quark, while a neutron consists of one up quark and two down quarks. This distinction accounts for the difference in mass and charge between the two particles. The quarks are held together by the strong nuclear force, which is mediated by gluons. The gluon is a member of the family of gauge bosons, which are elementary particles that mediate physical forces.

A quark is a generic type of physical particle that forms one of the two basic constituents of matter, the other being the lepton. Various species of quarks combine in specific ways to form protons and neutrons, in each case taking exactly three quarks to make the composite particle in question. There are six different types of quark, usually known as flavors: up, down, charm, strange, top, and bottom. The up and down varieties survive in profusion, and are distinguished by (among other things) their electric charge. It is this which makes the difference when quarks clump together to form protons or neutrons: a proton is made up of two up quarks and one down quark, yielding a net charge of +1; while a neutron contains one up quark and two down quarks, yielding a net charge of 0.

Quarks are the only fundamental particles that interact through all four of the fundamental forces. Antiparticles of quarks are called antiquarks. Isolated quarks are never found naturally; they are almost always found in groups of two (mesons) or groups of three (baryons) called hadrons. This is a direct consequence of confinement.

Atoms of the same element have the same number of protons, called the atomic number. Within a single element, the number of neutrons may vary, determining the isotope of that element. The total number of protons and neutrons determine the nuclide. The number of neutrons relative to the protons determines the stability of the nucleus, with certain isotopes undergoing radioactive decay. All known isotopes of elements with atomic numbers greater than 82 are radioactive.

About 339 nuclides occur naturally on Earth, of which 256 (about 76%) have not been observed to decay, and are referred to as “stable isotopes”. For 80 of the chemical elements, there is at least one stable isotope. Elements 43, 61, and all elements numbered 83 or higher have no stable isotopes. As a rule, there is, for each element, only a handful of stable isotopes, the average being 3.1 stable isotopes per element which has any stable isotopes.

Stability of isotopes is affected by the ratio of protons to neutrons, and also by presence of certain “magic numbers” of neutrons or protons which represent closed and filled quantum shells. These quantum shells correspond to a set of energy levels within the shell model of the nucleus; filled shells, such as the filled shell of 50 protons for tin, confers unusual stability on the nuclide. Of the 256 known stable nuclides, only four have both an odd number of protons and odd number of neutrons: hydrogen-2 (deuterium), lithium-6, boron-10, and nitrogen-14.

Every element has one or more isotopes that have unstable nuclei that are subject to radioactive decay, causing the nucleus to emit particles or electromagnetic radiation. Radioactivity can occur when the radius of a nucleus is large compared with the radius of the strong force, which only acts over distances on the order of 1 fm.

Most odd-odd nuclei are highly unstable with respect to beta decay, because the decay products are even-even, and are therefore more strongly bound, due to nuclear pairing effects. The most common forms of radioactive decay are alpha decay and beta decay.

Alpha decay is caused when the nucleus emits an alpha particle, which is a helium nucleus consisting of two protons and two neutrons. The result of the emission is a new element with a lower atomic number. Beta decay is regulated by the weak force, and results from a transformation of a neutron into a proton, or a proton into a neutron. The first is accompanied by the emission of an electron and an antineutrino, while the second causes the emission of a positron and a neutrino. The electron or positron emissions are called beta particles. Beta decay either increases or decreases the atomic number of the nucleus by one.

Another form of decay, Gamma decay, results from a change in the energy level of the nucleus to a lower state, resulting in the emission of electromagnetic radiation. This can occur following the emission of an alpha or a beta particle from radioactive decay.

Elementary particles possess an intrinsic quantum mechanical property known as spin. This is analogous to the angular momentum of an object that is spinning around its center of mass, although strictly speaking these particles are believed to be point-like and cannot be said to be rotating. Spin is measured in units of the reduced Planck constant (h), with electrons, protons and neutrons all having spin ½ h, or “spin-½”. In an atom, electrons in motion around the nucleus possess orbital angular momentum in addition to their spin, while the nucleus itself possesses angular momentum due to its nuclear spin.

The magnetic field produced by an atom—its magnetic moment—is determined by these various forms of angular momentum, just as a rotating charged object classically produces a magnetic field. However, the most dominant contribution comes from spin. Due to the nature of electrons to obey the Pauli exclusion principle, in which no two electrons may be found in the same quantum state, bound electrons pair up with each other, with one member of each pair in a spin up state and the other in the opposite, spin down state. Thus these spins cancel each other out, reducing the total magnetic dipole moment to zero in some atoms with even number of electrons.

In ferromagnetic elements such as iron, an odd number of electrons leads to an unpaired electron and a net overall magnetic moment. The orbitals of neighboring atoms overlap and a lower energy state is achieved when the spins of unpaired electrons are aligned with each other, a process known as an exchange interaction. When the magnetic moments of ferromagnetic atoms are lined up, the material can produce a measurable macroscopic field. Paramagnetic materials have atoms with magnetic moments that line up in random directions when no magnetic field is present, but the magnetic moments of the individual atoms line up in the presence of a field.

The nucleus of an atom can also have a net spin. Normally these nuclei are aligned in random directions because of thermal equilibrium. However, for certain elements (such as xenon-129) it is possible to polarize a significant proportion of the nuclear spin states so that they are aligned in the same direction—a condition called hyperpolarization. This has important applications in magnetic resonance imaging.

One area where natural elements, such as oxygen, may be hypothetically substantially different in substantially vast quantities, particularly at the subatomic level, is in the upper levels of the Earth's atmosphere. At great heights, such as in excess of 20 miles above sea level, the atmosphere is significantly different then the lower commercial, working, industrial levels of the atmosphere.

Because vast amounts of oxygen in the upper atmosphere are likely to have different subatomic properties than vast amounts of oxygen in lower levels, and because at the subatomic level there are properties and phenomena, and resultant affects on the human body, that are unexplainable and/or unknown, it is necessary and desirable to be able to study fluids, namely air, that may be captured from the upper atmosphere. There may be quantities of molecules in the upper atmosphere that have not been exposed to lower atmosphere environments. These molecules may have resided in the upper atmosphere undisturbed for billions of years. These molecules are affected significantly different than molecules near the Earth's surface. Gravity, radiation, molecule interaction, and lack of interaction with human conditions are all examples of different forces effecting like elements differently at the subatomic level. Because the elements are different at the subatomic level, the effect on the human body by these elements will be different.

An atmosphere is a layer of gases that may surround a material body of sufficient mass, such as the planet Earth, and are retained for a longer duration if gravity is high and the atmosphere's temperature is low. Surface gravity, the force that holds down an atmosphere, differs significantly among the planets. For example, the large gravitational force of the giant planet Jupiter is able to retain light gases such as hydrogen and helium that escape from lower gravity objects. Second, the distance from the sun determines the energy available to heat atmospheric gas to the point where its molecules' thermal motion exceed the planet's escape velocity, the speed at which gas molecules overcome a planet's gravitational grasp. Thus, the distant and cold Titan, Triton, and Pluto are able to retain their atmospheres despite relatively low gravities.

Since a gas at any particular temperature will have molecules moving at a wide range of velocities, there will almost always be some slow leakage of gas into space. Lighter molecules move faster than heavier ones with the same thermal kinetic energy, and so gases of low molecular weight are lost more rapidly than those of high molecular weight. Earth's magnetic field helps to prevent this, as, normally, the solar wind would greatly enhance the escape of hydrogen. However, over the past 3 billion years the Earth may have lost gases through the magnetic polar regions due to auroral activity, including a net 2% of its atmospheric oxygen.

The low temperatures and higher gravity of the gas giants—Jupiter, Saturn, Uranus and Neptune—allows them to more readily retain gases with low molecular masses. These planets have hydrogen-helium atmospheres, with trace amounts of more complex compounds. Two satellites of the outer planets possess non-negligible atmospheres: Titan, a moon of Saturn, and Triton, a moon of Neptune, which are mainly nitrogen. Pluto, in the nearer part of its orbit, has an atmosphere of nitrogen and methane similar to Triton's, but these gases are frozen when farther from the Sun. Other bodies within the Solar System have extremely thin atmospheres not in equilibrium. These include the Moon (sodium gas).

Referring now to FIG. 1, an illustration of the Earth's atmosphere is provided. The outermost layer of Earth's atmosphere, the Exosphere 1950, extends from the exobase upward. In the Exosphere 1950, particles are so far apart that they can travel hundreds of km without colliding with one another. Since the particles rarely collide, the atmosphere no longer behaves like a fluid. These free-moving particles follow ballistic trajectories and may migrate into and out of the magnetosphere 1991 or the solar wind. The exosphere 1950 is mainly composed of hydrogen and helium.

Another layer of the atmosphere is the thermosphere 1960, characteristic of where temperature increases with height in the thermosphere 1960 from the mesopause 1992 up to the thermopause 1993, then is constant with height. The temperature of the thermosphere 1960 may rise to 1,500° C. (2,730° F.), though the particles are so far apart that temperature in the usual sense is not well defined. The International Space Station orbits in the thermosphere, which is between 200 and 240 miles above the Earth. Between the top of the thermosphere 1960 and the bottom of the exosphere 1950 resides the exobase 1994. The elevation of the exobase 1994 varies with solar activity, and ranges from about 220 to 500 miles above the Earth. The Kármán line 1484, located within the thermosphere at an altitude of 62 ml, is commonly used to define the boundary between the Earth's atmosphere and outer space 1483.

The mesosphere 1970 extends from the stratopause 1995 up to about 50 to 53 miles above the Earth. The mesosphere 1970 is the layer where most meteors burn up upon entering the atmosphere. Temperature decreases with height in the mesosphere 1970. The mesopause 1992 has the temperature minimum of the mesophere 1970, and marks the top of the mesosphere 1970. This is generally considered the coldest place on Earth and has an average temperature around −100° C.

The stratosphere 1980 extends from the tropopause 1996 to about 170,000 feet (32 miles) above the Earth. Temperature increases with height in the stratosphere 1980, thereby restricting turbulence and mixing. The stratopause 1995 (i.e., the boundary between the stratosphere 1980 and mesosphere 1970) is typically in the range of 160,000 to 180,000 feet above the Earth. The pressure here is 1/1000th sea level.

The troposphere 1990 begins at the Earth's surface 1019 and extends to between 23,000 feet (7 miles) to about 56,000 feet (17 miles), with some variation due to weather. The troposphere 1990 is mostly heated by transfer of energy from the Earth's surface 1019, whereby on average the lowest part of the troposphere 1990 is warmest and temperature decreases with altitude. This promotes vertical mixing. The troposphere 1990 contains roughly 80% of the mass of the atmosphere. The tropopause 1996 is the boundary between the troposphere 1990 and stratosphere 1980.

The ionosphere 1997, the part of the atmosphere that is ionized by solar radiation, stretches from about 31 to 62 miles above the Earth, and typically overlaps both the exosphere 1950 and the thermosphere 1960. It forms the inner edge of the magnetosphere 1991. The ionosphere 1997 has practical importance because it influences, for example, radio propagation on the Earth.

The homoshpere 1998 and the heterosphere 1999 are defined by whether the atmospheric gases are well mixed. In the homosphere 1998, the chemical composition of the atmosphere does not depend on molecular weight because the gases are mixed by turbulence. The homosphere 1998 includes the troposphere 1990, stratosphere 1980, and mesosphere 1970. Above the Kaman line 1484 at about 330,000 ft (essentially corresponding to the mesopause), the composition varies with altitude. This is because the distance that particles can move without colliding with one another is large compared with the size of motions that cause mixing. This allows the gases to stratify by molecular weight, with the heavier ones such as oxygen and nitrogen present only near the bottom of the heterosphere 1999. The upper part of the heterosphere 1999 is composed almost completely of hydrogen, the lightest element.

The planetary boundary layer (“PBL”) 1948 is the part of the troposphere 1990 that is nearest the Earth's surface 1019, and is directly affected by the Earth's surface, mainly through turbulent diffusion. During the day, the PBL 1948 is usually is well-mixed, while at night the PBL 1948 becomes stably stratified with weak or intermittent mixing.

The average atmospheric pressure at sea level is about 1 atmosphere (atm)=101.3 kPa (kilopascals)=14.7 psi (pounds per square inch)=760 ton=29.9 inches of mercury (symbol Hg). Atmospheric pressure is the total weight of the air above unit area at the point where the pressure is measured. Thus air pressure varies with location and time, because the amount of air above the Earth's surface varies.

If atmospheric density were to remain constant with height the atmosphere would terminate abruptly at 8.50 km (27,900 ft). Instead, density decreases with height, dropping by 50% at an altitude of about 5.6 km (18,000 ft). As a result the pressure decrease is approximately exponential with height, so that pressure decreases by a factor of two approximately every 5.6 km (18,000 ft) and by a factor of e=2.718 . . . approximately every 7.64 km (25,100 ft), the latter being the average scale height of Earth's atmosphere below 70 km (43 ml; 230,000 ft). However, because of changes in temperature, average molecular weight, and gravity throughout the atmospheric column, the dependence of atmospheric pressure on altitude is modeled by separate equations for each of the layers listed above. Even in the exosphere, the atmosphere is still present. This can be seen by the effects of atmospheric drag on satellites.

50% of the atmosphere by mass is below an altitude of 5.6 km (18,000 ft). 90% of the atmosphere by mass is below an altitude of 16 km (52,000 ft). The common altitude of commercial airliners is about 33,000 ft and Mt. Everest's summit is about 29,029 feet above sea level. 99.99997% of the atmosphere by mass is below 330,000 ft (62 miles). The highest X-15 plane flight in 1963 reached an altitude of 354,300 ft.

The density of air at sea level is about 1.2 kg/m³ (1.2 g/L). Atmospheric density decreases as the altitude increases. Thus, the atmosphere becomes thinner and thinner with increasing altitude, with no definite boundary between the atmosphere and outer space.

As can be seen, the atmosphere, especially the upper levels of the atmosphere are affected by an abundance of factors, whereby the atmospheric constituents, especially at the subatomic level, may be significantly different than comparable constituent chemicals located near the Earth's surface (e.g., below 20 miles). As previously mentioned, the atmosphere is commonly referred to as air. Air is mainly composed of nitrogen, oxygen, and argon, which together constitute the major gases of the atmosphere. The remaining gases are often referred to as trace gases, among which are the greenhouse gases such as water vapor, carbon dioxide, methane, nitrous oxide, and ozone.

Many natural substances may be present in tiny amounts in an unfiltered air sample taken in the troposphere, including dust, pollen and spores, and volcanic ash. Various industrial pollutants also may be present, such as chlorine, fluorine compounds, mercury, and sulfur compounds such as sulfur dioxide. Blue light is scattered more than other wavelengths by the gases in the atmosphere, giving the Earth a blue halo when seen from space.

A sub-orbital flight may be a near-space flight in which the vehicle nearly reaches space, but its trajectory intersects the atmosphere or surface of the gravitating body from which it was launched, so that it does not complete one orbital revolution. By convention, the path of an object launched from Earth that reaches 62 miles above sea level, and then falls back to Earth, is considered a sub-orbital spaceflight, whereas an object that reaches an altitude less than 62 miles is a sub-orbital flight.

Some sub-orbital flights have been undertaken to test spacecraft and launch vehicles later intended for orbital spaceflight. Other vehicles are specifically designed only for sub-orbital flight; examples include manned vehicles such as the X-15 and SpaceShipOne, and unmanned ones such as ICBMs and sounding rockets.

The Kármán line 1484, was chosen by the Fédération Aéronautique Internationale because it is roughly the point where a vehicle flying fast enough to support itself with aerodynamic lift from the Earth's atmosphere would be flying faster than orbital speed. However, the US State Department does not support a distinct boundary between atmospheric flight and space flight.

If one's goal is simply to “reach space”, the lowest required delta-v is about 1.4 km/s, for a sub-orbital flight with a maximum speed of about 1 km/s. Moving slower, with less free-fall, would require more delta-v. Compare this with orbital spaceflights: a low Earth orbit (LEO), with an altitude of about 300 km, needs a speed around 7.7 km/s, requiring a delta-v of about 9.2 km/s.

For sub-orbital spaceflights covering a horizontal distance the maximum speed and required delta-v are in between those of a vertical flight and a LEO. The maximum speed at the lower ends of the trajectory are now composed of a horizontal and a vertical component. The higher the horizontal distance covered, the more are both speeds, and the more is the maximum altitude. For the V-2 rocket, just reaching space but with a range of about 330 km, the maximum speed was 1.6 km/s. SpaceShipTwo, or VSS Enterprise, which is under development by Virgin Galactic, will have a similar free-fall orbit but the announced maximum speed is 1.1 km/s (perhaps because of engine shut-off at a higher altitude).

It should be noted that any spaceflight that returns to the surface, including sub-orbital ones, will undergo atmospheric reentry. The speed at the start of that is basically the maximum speed of the flight. The aerodynamic heating caused will vary accordingly: it is much less for a flight with a maximum speed of only 1 km/s than for one with a maximum speed of 7 or 8 km/s.

A low Earth flight (LEF) is generally defined as a flight or orbit above the Karmen line up to an altitude of 1240 miles. Given the rapid orbital decay of objects below approximately 120 miles, the commonly accepted definition for LEF is between 100-1,240 miles above the Earth's surface. LEO is a low Earth orbit during a LEF around Earth between the atmosphere and below the inner Van Allen radiation belt. The altitude is usually not less than 1500 miles because that would be impractical as a result of larger atmospheric drag. With the exception of the lunar flights of the Apollo program, all human spaceflights have been either an LEF or have been sub-orbital.

Objects that undertake LEF encounter atmospheric drag in the form of gases in the thermosphere 1960 or exosphere 1950, depending on orbit height. Higher orbits include medium Earth orbit (MEO), sometimes called intermediate circular orbit (ICO), and further above, Geostationary orbit (GEO). Orbits higher than low orbit can lead to earlier failure of electronic components due to intense radiation and charge accumulation.

While a majority of artificial satellites are placed in LEO, where they travel at about 27,400 km/h (8 km/s), making one complete revolution around the Earth in about 90 minutes, many communication satellites require geostationary orbits, and move at the same angular velocity as the Earth.

Since it requires less energy to place a satellite into a LEO and the LEO satellite needs less powerful amplifiers for successful transmission, LEO is still used for many communication applications. Because these LEO orbits are not geostationary, a network (or “constellation”) of satellites is required to provide continuous coverage. Lower orbits also aid remote sensing satellites because of the added detail that can be gained. Remote sensing satellites can also take advantage of sun-synchronous LEO orbits at an altitude of about 500 ml and near polar inclination.

Atmospheric and gravity drag associated with launch typically adds 1,500-2,000 m/s to the Delta-V launch vehicle required to reach normal LEO orbital velocity of around 7,800 m/s (17,448 mph).

The cost to run large scale government, corporate, and/or private projects that involve suborbital spaceflight or LEF are substantial and prohibitive. Currently, vehicles such as satellites, space shuttles, and the like must be launched to high altitudes by rockets. This requires enormous expenditure of fuel, and, absent very high geosynchronous orbit, do not maintain position relative to the earth. Thus, operation performed from these vehicles, such as deployment of satellites carrying communication devices or signaling systems is a very expensive proposition, and is often not cost-effective.

In 1960 the NASA space agency consisted of a small headquarters in Washington, its three inherited NACA research centers, the Jet Propulsion Laboratory, the Goddard Space Flight Center, and the Marshall Space Flight Center. With the advent of Apollo, these installations grew rapidly. In addition, NASA added three new facilities specifically to meet the demands of the lunar landing program. In 1962 it created the Manned Spacecraft Center (renamed the Lyndon B. Johnson Space Center in 1973), near Houston, Tex., to design the Apollo spacecraft and the launch platform for the lunar lander. This center also became the home of NASA's astronauts and the site of mission control. NASA then greatly expanded for Apollo the Launch Operations Center at Cape Canaveral on Florida's eastern seacoast. The cost of this expansion was great, more than 2.2 billion over the decade, with 90 percent of it expended before 1966. This was an enormously expensive and time-consuming process. The resultant waste of governmental programs has not subsided.

In 2004, President Bush launched the Constellation project with the goal of returning to the moon by 2020 and then establishing a lunar launch pad for a first trip to Mars. However, the space community has always complained that NASA's annual budget of $18 billion was inadequate for the purpose. Moreover, after allocation of billions of dollars for the project, the Constellation project has been recently terminated.

Because of the enormous cost associated with attaining sub-orbital space flight and LEF, it is not practical for small capital projects or operations to be pursued.

As a result, the current Executive branch regime has encouraged private industry to pursue space and near-space projects. Some examples include Rocketplane Limited and Blue Origin, which are taking an interest in sub-orbital spaceflight as a result of ventures like the Ansari X Prize. In addition, the VSS Enterprise is expected to begin manned sub-orbital spaceflights starting in 2011.

Typically, researchers wish to conduct experiments in microgravity or at high atmosphere altitudes. There is a need for a cost effective above-surface apparatus that may be used to perform research and experimentation at the sub-orbital level.

It is known to use airships or dirigibles for low altitude applications, such as surveillance, signaling, collecting and transmitting meteorological data, and the like. These are typically permanently tethered to a fixed ground location and are deployed in the lower atmosphere.

Several schemes have been used in attempts to collect air samples in the low altitudes, which include towers and tethered balloons. Tethered balloons, for example, provide flexibility to easily vary deployment areas, and have the advantage of being able to reach 1,000 meters. However, one of the major limitations of tethered balloons is that they do not reach high altitude, and they are tethered.

Thus, there is a strong need to pursue operations with LLEF. As admitted by a NASA spokesman, operations performed by LLEF like that of the present disclosure would have cost NASA and taxpayers millions of dollars. A typical space shuffle mission flies 200 miles above the earth's surface and returns beautiful pictures on the way, but it involves 1,500 people, puts six or seven astronauts at risk and costs, and can cost up to half a billion dollars.

Thus, the research as it pertains to upper level atmospheric constituents, and their subsequent effect on humans, may not proceed unless a viable, economic ability is provided for.

What is needed is a vehicle capable of atmospheric flight that is safer, reliable, and more affordable.

There is a great need for the processing of a fluid, or multiple fluids, in order produce an oxygenated fluid. The resultant oxygenated fluid may be a restorative drink saturated with molecular oxygen. This may occur by increasing the oxygen content of the drink by saturation. There is a need to obtain atmospheric fluids that reside at elevations 20,000 feet above sea level.

### SUMMARY OF INVENTION

In one aspect, embodiments disclosed herein relate to a sub-orbital lower-level apparatus for collecting an atmospheric fluid, the apparatus including a launch device configured for overcoming resistive forces in order to reach a layer of Earth's atmosphere that resides at a predetermined altitude greater than 20,000 feet above sea level, and a fluid capture mechanism operatively connected to the launch device and configured to acquire and store an atmospheric fluid.

In another aspect, embodiments disclosed herein relate to a method for oxygenating a fluid, the method including launching a sub-orbital lower-level apparatus to a predetermined altitude greater than 20,000 feet above sea level. The method also includes, at the predetermined altitude, actuating a fluid capture mechanism sealingly configured to collect and store an atmospheric fluid. In addition, returning the a sub-orbital lower-level to an altitude less than 20,000 feet, and introducing at least a portion of the atmospheric fluid to an oxygenation process to mix the captured atmospheric fluid with another fluid.

Further, in yet another embodiment, embodiments disclosed herein relate to a method of operating a sub-orbital lower-level apparatus, the method including launching the sub-orbital lower-level apparatus to a layer of the Earth's atmosphere located at predetermined altitude greater than 20,000 feet above sea level. The sub-orbital lower-level apparatus includes a two-stage booster device further having a first stage and a second stage, wherein the first stage is configured with a non-combustible lighter-than-air material to launch the apparatus through a first predetermined layer of the atmosphere, and wherein the second stage has an ignitable material configured to maneuver the apparatus in a second predetermined layer of the atmosphere, and wherein the layer resides within the second predetermined layer. The method further includes performing an operation with the sub-orbital lower-level apparatus at the predetermined altitude, and returning the sub-orbital lower-level apparatus to an altitude less than 20,000 feet.

Other aspects and advantages of the invention will be apparent from the following description and the appended claims.

### BRIEF DESCRIPTION OF DRAWINGS

FIG. 1 shows a view of an illustration of the Earth's atmosphere.

FIGS. 2A, 2B, 2C, and 2D show multiple views of various sub-orbital lower-level Earth vehicles, in accordance with embodiments disclosed herein.

FIGS. 3A and 3B show a functional block diagram of a fluid processing system and a macro view of the fluid processing system, in accordance with embodiments disclosed herein.

FIGS. 4A, 4B, and 4C show multiple views of a fluid processing system, in accordance with embodiments disclosed herein.

### DETAILED DESCRIPTION

Specific embodiments of the present disclosure will now be described in detail with reference to the accompanying Figures. Like elements in the various figures may be denoted by like reference numerals for consistency. Further, in the following detailed description of embodiments of the present disclosure, numerous specific details are set forth in order to provide a more thorough understanding of the invention. However, it will be apparent to one of ordinary skill in the art that the embodiments disclosed herein may be practiced without these specific details. In other instances, well-known features have not been described in detail to avoid unnecessarily complicating the description.

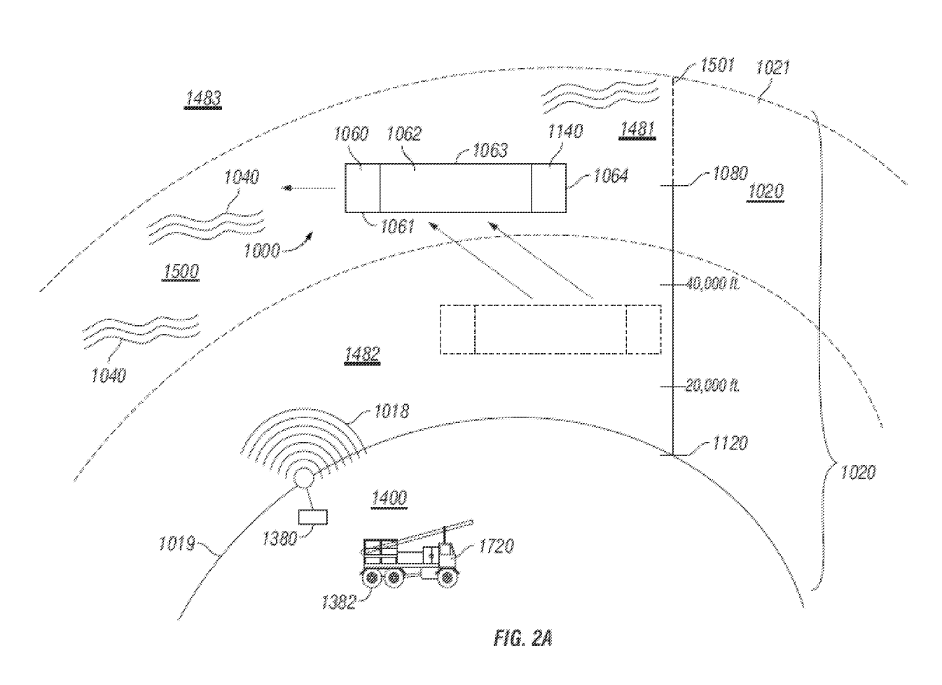

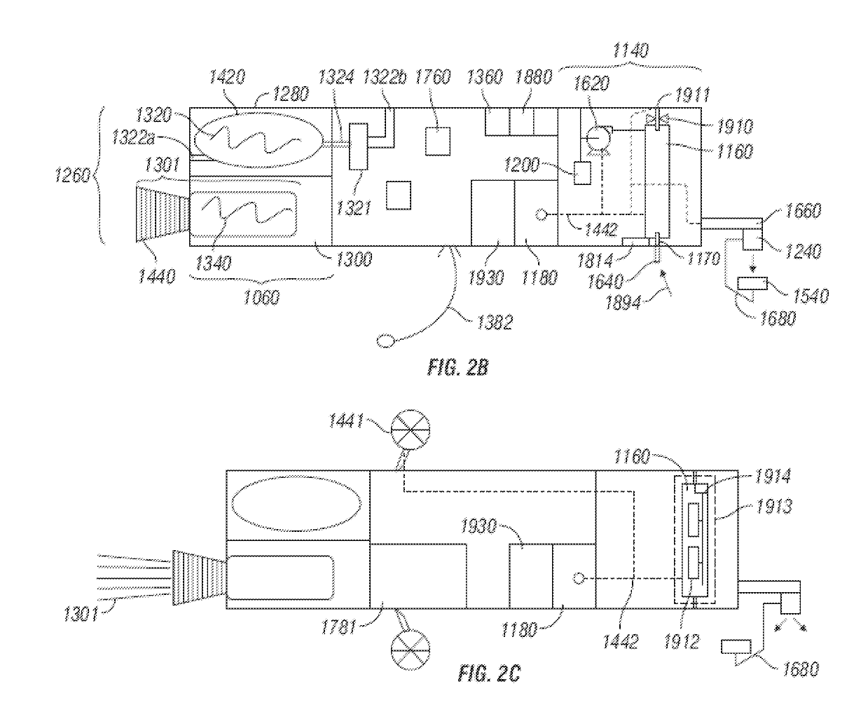

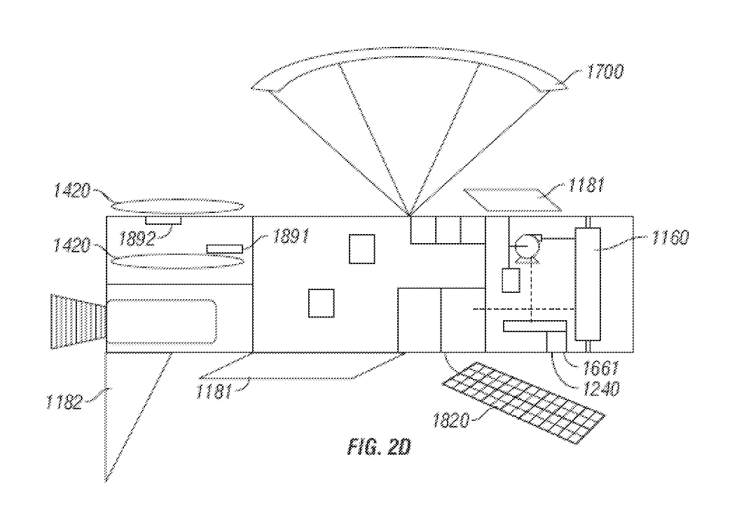

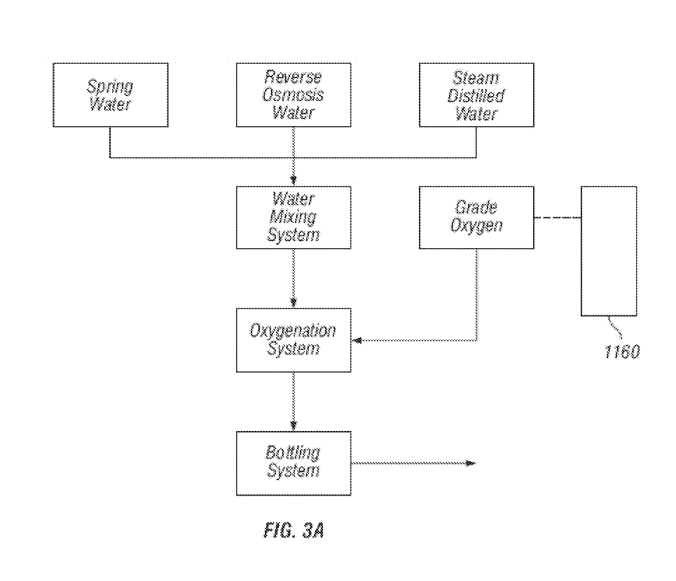

Referring now to FIGS. 2A, 2B, 2C, and 2D, multiple views of comparable sub-orbital lower-level Earth vehicles 1000 in accordance with embodiments disclosed herein, are shown. Lower-level Earth flight (LLEF) may be a suborbital flight that occurs at altitudes higher than commerical airliner airspace, but lower than LEO airspace. Thus, LLEF may occur at elevations above the Earth's surface 1019 greater than 20,000 feet, but less than 100 miles. FIGS. 2A-2D together illustrate an above-surface apparatus, which may have slight variations in configurations and accompanying components, and hereinafter referred to as a sub-orbital lower-level Earth vehicle (“SOLLEV”) 1000, may be used to perform operations during LLEF.

As an example of one operation, the SOLLEV 1000 may be used for collection of an atmospheric fluid 1040. The SOLLEV 1000 may include a launch device 1060 configured to actuate, thereby providing the apparatus 1000 with the ability to overcome resistive forces in order to reach a layer of Earth's atmosphere 1500 that resides at a predetermined altitude 1080 greater than 20,000 feet above sea level 1120. There may be a fluid capture mechanism (hereinafter “FCM”) 1140 operatively connected to the apparatus 1000, and the FCM 1140 may be configured to acquire, capture, and store the atmospheric fluid 1040.

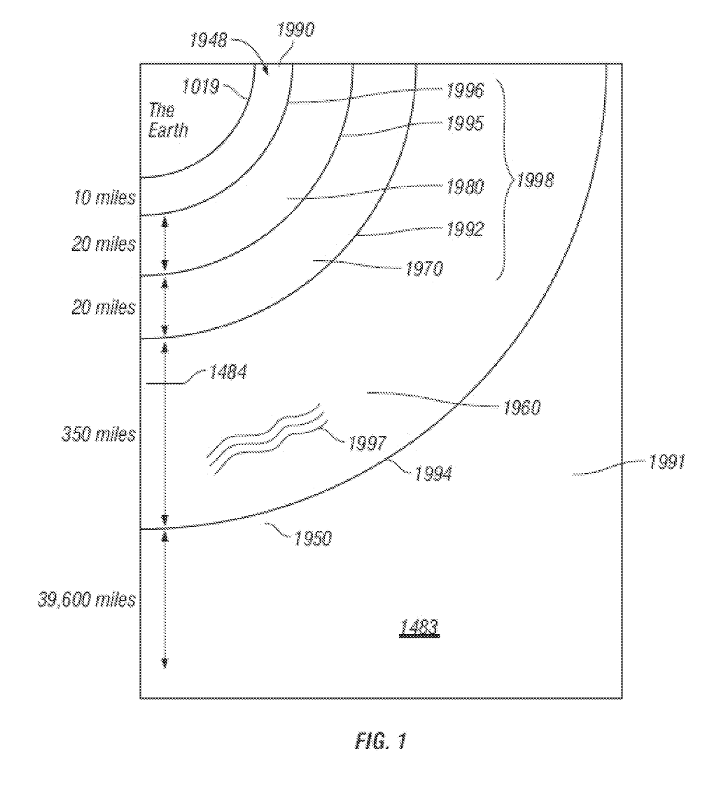

As an example of another operation, the SOLLEV 1000 may be configured to take a camera 1240 high into the atmosphere, such as into the stratosphere (1980, FIG. 1) in order to take pictures of the Earth and/or atmosphere. The camera 1240 may be operatively enclosed and/or mounted in a pressurized compartment, such as the main body 1062.

The launch device 1060 may include a housing 1061 connected to a main body 1062 of the SOLLEV 1000. Components of the SOLLEV, including, for example, the launch device 1060, the housings 1061, 1062, 1063, etc., may be configured to withstand extreme pressure and temperature conditions not normally associated with near-sea level elevation. Thus, the SOLLEV 1000, which may be made from materials of construction, such as reinforced steel or reinforced fiberglass, may be configured to withstand near-vacuum pressure absolute and/or temperatures that range from −100° C. to 200° C.

FIG. 2B illustrates a close-up view of the SOLLEV 1000, according to embodiments disclosed herein. The launch device 1060 may include a two-stage booster section 1260, such that the launch device 1060 may have a first stage 1280 and a second stage 1300. The first stage 1280 may be configured with, for example, a lighter-than-air (“LTA”) material 1320 disposed within an expandable balloon 1420 and/or LTA supply tank 1321. The LTA 1320 may allow the SOLLEV 1000 to launch and/or rise above sea level 1120 to a first predetermined layer 1482 of the atmosphere 1020.

The second stage 1300 may be configured with, for example, a combustible material 1340 that may be used in such a way as to allow the SOLLEV 1000 to freely maneuver while above the Earth's surface 1019. In one embodiment, the combustible material 1340 may facilitate the maneuvering while the SOLLEV 1000 is in a second predetermined layer 1481 of the atmosphere 1020. In a further embodiment, the layer 1500 may reside within the within the second predetermined layer 1481.

In another embodiment, the two-stage booster 1260 may have the first stage 1280 configured with a non-combustible material, the use of which may be comparable to the LTA material, such that the non-combustible material may allow the SOLLEV 1000 to launch above sea level 1120 upward through the first predetermined layer 1482. The booster 1260 may also have the second stage 1260 configured with a propellant material, the use of which may allow the SOLLEV 1000 to maneuver while in a predetermined layer 1481, 1482, and/or 1500 of the atmosphere. In an exemplary embodiment, the propellant material may be used to maneuver the SOLLEV 1000 while in the layer 1500.

The position of the layer 1500, and hence where the SOLLEV 1000 may perform above-surface operations, is not meant to be limited. As one example, the layer 1500 may reside within either the first predetermined layer 1482 or the second predetermined layer 1481. As another example, the layer 1500 may reside at a predetermined altitude greater than 40,000 feet. As yet other examples, the layer 1500 may reside at an altitude in the range from about 40,000 feet to an altitude 1501 that is substantially adjacent the Earth's atmosphere and comprises a natural vacuum (i.e., substantially zero pressure absolute), or the layer 1500 may reside at a predetermined altitude greater than 40,000 feet.

The launch device 1060 may include other additional components and subcomponents. For example, the first stage 1280 may also include a balloon 1420. The LTA 1320 may include a non-combustible gas, such as, for example, helium gas. As another example, the second stage 1300 may also include at least one booster engine 1301. The booster engine 1301 may be configured to exhaust a gas stream 1341, which may result from ignition of the combustible material 1340.

In one embodiment, the above-surface vehicle 1000 may include deployment of the inflatable and/or expandable balloon 1420 in order to facilitate the SOLLEV's ability to break free of and/or overcome resistive forces, such as gravity, friction, drag, etc. The balloon 1420 may be filled with LTA 1320, such as helium or the like. In one embodiment, the SOLLEV 1000 may reach a pre-determined altitude 1080 above sea level 1120. For example, the SOLLEV 1000 may be configured to reach a predetermined altitude of 20,000 feet. As another example, the predetermined altitude 1080 may be in the range of about 20,000 feet to 65,000 feet.

The SOLLEV 1000 may include the inflatable balloon 1420 configured to be filled with the LTA material 1320, and may further be configured to rise to a predetermined altitude 1080. The SOLLEV 1000 may be controllable without tethering to the ground once the inflatable balloon 1420 is filled with a sufficient amount of LTA material 1320. Once deployed, the SOLLEV 1000 may rise to the higher operating altitude.

In one embodiment, the SOLLEV 1000 may be configured to rise to an altitude of more than 40,000 feet. In another embodiment, the SOLLEV may be configured to perform operations while in the altitude range from 65,000 to 85,000 feet. The SOLLEV 1000, and associated components thereof, may be made from very high strength, flexible, and lightweight material.

The overall shape of the SOLLEV 1000 is not meant to be limited. But for an example and for description purposes, the vehicle 1000 may be an elongated, aerodynamic shape similar to that of a conventional blimp.

The SOLLEV 1000 size and/or volume may be sufficient to lift the required weight and/or payload to the predetermined altitude 1080. In an embodiment, the SOLLEV 1000 may be configured to have a positive buoyancy in the range of 0 to 10,000 lbs. Thus, the inflatable balloon 1420 may be filled with a sufficient amount of gas so that the SOLLEV 1000 may have enough buoyancy to lift a load in the range of 0 to 10,000 lbs about the Earth's surface 1019.

A controlled supply tank 1321 may be provided within the SOLLEV 1000, along with a vent system 1322, which may include vent portions 1322a and 1322b. The vent system(s) may be used to provide controlled venting for buoyancy adjustment of the SOLLEV 1000.

As mentioned, the SOLLEV 1000 may include a propellant rocket engine 1301. In one example of operation, liquid propellant combinations embodying oxidizers and fuels which have been considered are liquid oxygen(LOX)/kerosene and liquid oxygen (LOX)/liquid hydrogen (LH2) engines; LOX/methane and LOX/LH2 engines; and solid propellant motors and LOX/LH2 engines may be used to provide the thrust necessary to maneuver in the atmosphere.

The SOLLEV 1000 may also include, other components, such as the aforementioned balloon 1420. The balloon 1420 may be adapted to be filled with a suitable, LTA material 1320, such as helium.

In addition to engine 1301, there may be other maneuvering devices, such as jet engines (not shown), thrusters 1441, etc., any of which may operatively mounted to the SOLLEV 1000. These devices may be used for positioning, maneuvering, launching, etc. The SOLLEV may include other sensors to facilitate operations, such as temperature, pressure, and altitude, which may all be maintained in the launch device (flight box).

The thruster 1441 may, for example, be connected to the power source 1180, or may use some other form of energy, such as solar power. Thus, the SOLLEV may be configured with solar panels 1820 (retractable or otherwise) and associated electrical circuitry 1442 for power collection and provision purposes. Although circuitry 1442 is not illustrated as connected to each and every component, it would be obvious to one of ordinary skill in the art that any and all components that require a power source may be operatively connected to power source 1180 with circuitry 1442.

A controlled gas supply may flow between the supply tank 1321 and the balloon 1420. In addition and/or alternatively to, the vent system 1322 also may be used to remove, or otherwise adjust, the amount of gas, the buoyancy, the lift, etc. of the SOLLEV 1000.

Operation of the system will now be described in more detail with reference to the Figures that help illustrate launching the SOLLEV 1000 to a predetermined altitude 1080. The SOLLEV 1000 may initially rise above the Earth surface 1019 as a result of the first stage 1280 propulsion/bouyancy, which may include controlled inflation of balloon 1420 with a controlled amount of LTA material 1320 (e.g., gas). Alternatively, the balloon 1420 may be initially inflated with gas from a supply tank disposed on a launch recovery vehicle (“LRV”) 1720, such that the balloon 1420 may be directly filled with LTA material 1320 from the LRV 1720. In this aspect, when the balloon 1420 is inflated to a predetermined amount, the SOLLEV 1000 may be released, and will immediately start to rise above the Earth's surface 1019.

The launch of the SOLLEV 1000 may be controlled as to time and location to avoid jet streams, air traffic, or any other man-made or natural phenomena that is detrimental to the operation, launch, etc.

Once the SOLLEV 1000 rises above a pre-determined layer 1482 of the atmosphere and begins to reach a pre-determined altitude 1080, the buoyancy provided by the first stage 1280 may be controlled (or otherwise maintained) or stopped in order to control altitude. At the predetermined altitude 1080, operations may be performed, which may include, for example, actuation of the second stage 1060 (i.e., the booster engine 1301), actuation of the thrusters 1441, actuation of the FCM 1160, taking a picture with the camera 1240, etc.

The SOLLEV 1000 may thus be deployed and launched in order to perform various operations at significant elevations above the Earth's surface 1019. At the pre-determined altitude, which may be lower than a second predetermined layer 1481 of the atmosphere, the thrusters 1441 may be used to maintain proper position or to change the position. The SOLLEV 1000 may have a remote control positioning system 1762 and/or an auto-pilot system 1721 for positioning. This may be, for an example or in addition to, a GPS 1760 usable for accurate positioning.

Once launched, the SOLLEV may use communications system/devices 1381 for sending and/or receiving transmissions, such as to and from the surface 1019. The SOLLEV 1000 may be equipped with other operations equipment, such as one or more cameras 1240. The camera 1240 may be used for optical or infra red surveillance purposes, or for taking pictures.

The SOLLEV 1000 may be manned or unmanned. If manned, there may be a pressurized cabin 1781 disposed in the main body 1062, which may be configured for one or more passengers (not shown). As such, the SOLLEV 1000 may be used for recreational purposes, and people may be transported to and from locations on the ground. Robotic and/or other kinds of automated systems may be provided for in the SOLLEV 1000, such as a robotic mechanical arm 1660. The mechanical arm 1660 may be used to facilitate operations, such as taking an image or making repairs to equipment after launch.

It may be necessary for the SOLLEV 1000 to be operational for extended periods of time. Thus, the SOLLEV 1000 may include devices that provide for retrieval of any associated equipment, as well as external equipment. For example, there may be a second smaller mini-SOLLEV (not shown) suspended in the layer 1500 that requires retrieval. The recovery system of this invention is inexpensive, easy to operate, and convenient.

To begin descent or landing, it may be necessary to deflate the balloon 1420, either partially or in entirety. This may be done, for example, by using the vent system 1322; however, there are any number of ways to invoke or begin descent of the SOLLEV 1000, and how the balloon 1420 is defalted is not meant to be limited by the disclosure. For example, the balloon 1420 may be deflated by tearing, rupturing, explosion, or over-inflation.

During the descent of the SOLLEV 1000, the LRV 1721 may use a communication device, such as a transreceiver 1382, to track signals from transponder or other transmitter 1880 mounted on the SOLLEV 1000.

The SOLLEV 1000 may be equipped with devices that provide for accurate and reliable tracking, monitoring, etc. of the SOLLEV 1000. Receiving antennae 1382 or the like may be used to help with GPS positioning purposes. In addition, an automatic control system 1761 may be used to actuate and/or control devices, such as the engines, jets, vent system, etc. in order to move (i.e., maneuver, (re)position, etc.) the SOLLEV 1000 to any desired location based on its detected position.

As mentioned, the SOLLEV 100 may be provided with the ability to carry out many additional operations not presently possible with other LEO vehicles like satellites or the like. In operation, the SOLLEV 1000 may be in flight toward the predetermined altitude 1080. At the altitude 1080, the FCM 1140 may be activated to collect a sample of the atmosphere 1040, for which the sample may be used for analytical and/or experimental purposes. Once the predetermined altitude 1080 is reached, the amount of LTA material 1320 in the balloon 1420 may be adjusted in order to maintain the predetermined altitude 1080.

The SOLLEV 1000 may be configured with, for example, loft insulation that may be used to wrap the camera 1240, the GPS, and other components and/or digital equipment from harsh temperatures that may reach freezing temperatures of −60° C. (−75° F.). The balloon 1420 may be a helium balloon configured to lift the SOLLEV 1000 above the Earth's surface 1019. The balloon 1420 may expand to a diameter of up to 20 meters, before popping and letting the SOLLEV 1000 fall back to Earth via an attached parachute 1700.

In other aspects, the balloon 1420 may be deflated and/or collapsed. For example, when that altitude 1080 is reached, and operations are performed and subsequently completed, then a suitable pressure-sensitive switch (“PSS”) circuit 1891, which may include a power source (e.g., batteries) and a squib operably connected with the PSS. The PSS circuit 1891 may be configured to allow firing of the squib as would be known to one of ordinary skill in the art, whereby firing of the squib may sever the balloon 1420.

Upon severance, and pursuant to and by reason of rapid descent of the SOLLEV 1000, atmospheric fluid 1040 may be rammed as indicated by the arrows 1894 into the pitot tube 1640, and through the distribution network 1913 into at least one of the sealed chambers 1912. The rammed fluid may continue to fill into other sealed chambers as determined by the controller and a control valve (not shown), or until the SOLLEV 1000 descent is stopped or limited.

The capture sequence may be programmed to stop after a predetermined event occurs, such as time, elevation (or descent), etc. For example, the filing of the sealed chamber 1160 may have been pre-calculated to be substantially complete, whereby another suitable pressure-sensitive switch circuit 1894 (like the circuit 1891 and/or 1892) actuate to terminate the sequence, such as by firing in order to cause the chamber 1912 to be completely closed and sealed off.

There may be additional pressure-sensitive switch circuits (not shown), which may also be in electrical communication with a power source, such as power source 1180. For example, there may be a flight termination squib, which may be used as a back-up or fail safe in order to prevent the SOLLEV 1000 from reaching a dangerously too-high altitude on accident. Upon activation and/or firing of the termination squib, the SOLLEV 1000 may be severed or released from the balloon, and/or the fill line 1324 may be severed in order to stop filling of the balloon 1420. Afterward, the SOLLEV 1000 may commence descent.

In an embodiment, a parachute 1700 connected to the SOLLEV 1000 may be deployed outwardly, and the parachute 1700 may open allow the SOLLEV 1000 to gravitate gently to the surface 1019. Thus, the use of a parachute 1700 to control descent and/or landing may prevent the SOLLEV 1000 and any components from being damaged upon landing.

While the description above mentions squibs and pressure-sensitive switches, other devices, such as a timer mechanism or other remote device, may be used to control actions on the device, such as the severing. For example, the timer, after a pre-determined period would operate to close a switch, whereby a motor actuates to actuate another device(s).

The timer may, for example, be started pursuant to closing of the motor starting switch and could be set to close the switch at a predetermined subsequent time, to fire the squib 78. Likewise, safety squib could be timer controlled instead of altitude-controlled. It should be readily apparent that other means could be used to begin descent.

The balloon(s) and other devices on the SOLLEV may be formed of various types of materials, including those that are lightweight, low temperature tolerant, and resistant to rupture at high altitudes, during descent, and landings. For example, the materials could be polyethylene.

The SOLLEV 1000 may include a sealed chamber 1160 disposed in the FCM 1140, whereby the FCM 1140 may be able to sealingly store and/or retain the captured atmospheric fluid 1040. In one embodiment, the atmospheric fluid 1040 may include gaseous atmospheric air. In another embodiment, the atmospheric fluid 1040 may include appreciable amounts of water vapor. There may be an actuator 1200 that is remotely operable, and may be configured to actuate the FCM 1140 at a predetermined time and/or altitude.

The actuator 1200 may be remotely operable via a timer mechanism 1360 that may be configured to operate at a predetermined time. However, the actuator 1200 may be remotely operable in other fashions, such as via a wireless transmission. The wireless transmission may be a signal from a satellite 1021, or any other kind of transmitter, such as the transceiver 1380 that transmits a wireless signal 1018. In one embodiment, the wireless transmission 1018 may be initiated from other devices, such as, for example, from a cell phone or a computer (not shown).

The FCM 1140 may have components mounted internally or externally to the SOLLEV 1000. The FCM 1140, in conjunction with the SOLLEV 1000, may be used to collect samples of fluids, such as atmospheric air. The FCM 1140 may be used to provide means to capture previously irretrievable portions of the atmosphere for study, such as to establish the vertical homogeneity of trace gas composition of the atmosphere at high altitudes. Although not illustrated, there may be more than FCM 1140 that may be used, including simultaneously.

As illustrated, there may be a pump 1620, such as a vacuum pump, configured to move atmospheric fluid near the inlet 1911 past a control valve 1910 and into at least one of sealed chamber 1912. In an embodiment, the FCM 1140 may include a plurality of sealed chambers 1912 disposed therein.

Thus, the FCM 1140 may be used to collect fluids, such as gases, from the atmosphere. As shown, the FCM 1140 may be connected to the SOLLEV 1000 by way of a mounting plate 1913. The FCM 1140 may have a number of components associated therewith. There may be an inlet 1911 in fluid communication with a pump 1620.

The pump 1620 may be used to draw a fluid, such as a gas, which may be atmospheric air, into the inlet 1911, and into sealed chamber 1912. The FCM 1140 may direct and flow fluids along flow line 1914, and past control valve 1910. The flow line 1914 may be part of the distribution network 1913. The control valve 1910, or other valves (not shown), may be used to determine the direction the inlet gas goes, including through the distribution network 1913, which may direct the fluid to at least one, or more, sealed chamber(s) 1912 for storage. Thus, the sealed chamber 1160 may have a plurality of additional sealed chambers 1912 disposed therein.

The SOLLEV 1000, as well as any of the operational systems on board, may be in radio communication with a transceiver (i.e., transmitter-receiver) 1380. Thus, a wireless device or the like may be used to provide data about the operation of the FCM 1140, or other operations performed by the SOLLEV 1000.

A power source 1180 may be used and configured to provide electrical power to the FCM 1140, or any other component operatively connected to the SOLLEV 1000. Thus, all components on the SOLLEV 1000 may be operatively connected to the power source 1180 by any circuitry (or other circuitry, such as electrical, fiber optic cable, infrared, wireless, etc.). As but one example, the power source 1180 may include a plurality of batteries, such as 12 AA size nickel cadmium rechargeable batteries.

A programmable controller 1930 may be used to receive signals, data, or other information from the transceiver 1380. Thus, the controller 1930 may be used to operate the FCM 1160, the actuator 1200, the control valve 1910, the pump 1620, etc.

The FCM 1140 may include one or more pitot tubes 1640 mounted externally to the FCM 1140, such that a fluid (i.e., air, etc.) may be rammed or forced into the inlet of the pitot tube 1640, through the distribution network 1913, and into a sealed chamber 1160 or chambers 1912. The sealed chamber 1160 of the FCM 1140 may be configured with a number of one-way flow valves (not shown), such as a check valve, to prevent stored gas from leaving the sealed chamber 1160.

The SOLLEV 1000 may include a power source 1180 configured to provide power to the SOLLEV 1000 and any components associated thereof, whereby the components are operatively connected with the power source, such as by electrical circuitry 1442, or any other operable circuitry known to one of ordinary skill in the art as may be needed to provide power to each and every component of the SOLLEV 1000.

A suitable power source 1180 may be provided in the SOLLEV 1000 in order to provide power for any onboard operations, such as to operate/ignite engines (or other propulsion or maneuvering devices), or actuate the mechanical arm 1660. The power source 1180 may include, for example, one or more of batteries, a stored fuel supply, solar power, microwave power, and any combinations thereof.

The SOLLEV 1000 and/or the LRV 1721 may use the GPS 1760 disposed on the SOLLEV 1000 to provide accurate positioning or station-keeping purposes, as well as to keep the SOLLEV 1000 at the pre-determined position 1081 or altitude 1080, within acceptable limits.

Rudders (not shown), elevators 1182, or any other kind of fin 1181 may also be operatively connected to the SOLLEV 1000 by linkages and actuators, such as mechanical linkage and mechanical, pneumatic, hydraulic etc. actuators. The camera 1240 may be mounted to the SOLLEV, such that the camera 1240 is internal, but may take external images through window 1661.

The SOLLEV 1000 may include an onboard camera 1240 operably connected therewith that may be configured to take an image 1220. In one embodiment, the camera 1240 may take an image while the SOLLEV 1000 is in the layer 1500. The camera 1240 may be configured to take an image while the SOLLEV 1000 is maneuvering in the layer 1500. The image may include, for example, a second image 1540 disposed in the viewing range of the camera 1240.

The camera 1240 may also be configured to take pictures in various orientations and/or directions. The camera 1240 may be any camera, as would be known to one of skill in the art. The camera 1240, which may be a digital camera, may be preprogrammed to sleep (e.g., hibernate) and reactivate at predetermined intervals. Similarly, the FCM 1160 may be preprogrammed in a comparable manner. The camera 1240 may take images of the atmosphere for scientific study, which may include the curvature of the earth, the blue band of the atmosphere, and the blackness of space.

A suitable launch/retrieval vehicle (LRV) 1720 may provide applicable ground control operations, such as the ability to position the SOLLEV 1000 prior to and/or during launch, provide LTA material for balloon inflation, and also for tracking and retrieving the SOLLEV 1000. The LRV 1720 may include a suitable receiver 1382 used to track, for example, signals emitted by the transmitter, transponder, etc. on the SOLLEV 1000. The SOLLEV may use a Global Positioning System (GPS) 1760 or other techniques used for tracking purposes. The SOLLEV 1000 may be designed to be manned or unmanned. Thus, the SOLLEV 1000 may have a pressurized passenger cabin 1781 for transporting personnel.

Communication devices 1381 such as receivers, transmitters, transponders, antennae, etc., which may be of the type conventionally installed in communications satellites or other airborne vehicles, may be mounted internal and/or external to the SOLLEV 1000. These may comprise, for example, RF communication link devices for television, radio, portable phones, microwaves, and the like. The comm devices 1381 may be to determine the current position of the SOLLEV 1000. The devices 1381 may also be used to determine the location of the SOLLEV 1000 after return to the Earth.

The SOLLEV 1000 may contain various control systems, such as environmental, electronic, and engine controls (not shown). There may be a retractable payload door 1860 that encloses a payload or a cargo compartment 1780. The comm devices 1381 may be suitably mounted as necessary internally or externally of the SOLLEV 1000. The comm devices may be used to provide for communications such as television, radio, or other wireless signals and the like.

The camera 1240 may be controlled by a controller, CPU, microprocessor, microcomputer, etc., such that the camera 1240 may take pictures at timed intervals. The controller may be, for example, an ATMega8. The ATMega8 is an excellent microchip for this kind of work, which may be programmed, for example, by Linux, Windows or the Mac software/hardware. Gumstix (Gumstix Verdex) develops and sells small, inexpensive, highly functional Linux computers for outstanding development and production systems usable with embodiments of the disclosure.

The GPS may be, for example, a GSM/GPS, such as a Timble Lassen IQ or Trimble Lassen SK II. The GPS may be used to track the SOLLEV and/or aid recovery of any payload. These GPS may be restricted from operation should a certain speed and/or altitude be exceeded, but as long as these restrictions are not exceeded the GPS keeps functioning.

The communications device(s) may be those provided by, for example, Radiometrix. This company specializes in the design and manufacture of low power radio products for rapid implementation of high-reliability, cable-free data links. Radiometrix is the industry's leading developer of off the-shelf, license-exempt miniature radio modules that is suitable for LLEF.

Sensor devices may include, for example, sensors provided by Dallas Semiconductor (now subsidiary of Maxim-ic). One example of a sensor is a low temperature sensor, which may operate in the temperature range is from −55 deg C. to 150 deg C. making it a good choice for high altitude operations.

When the SOLLEV returns to the Earth's surface, any fluid captured by the FCM 1140 may be used further. For example, the fluid may be studied or analyzed. As another example, the fluid may be used to process another fluid. In one embodiment, the fluid may be separated into constituent components. For example, if the fluid is atmospheric air, the air may be separated into a purified oxygen stream, a nitrogen stream, and a waste stream.

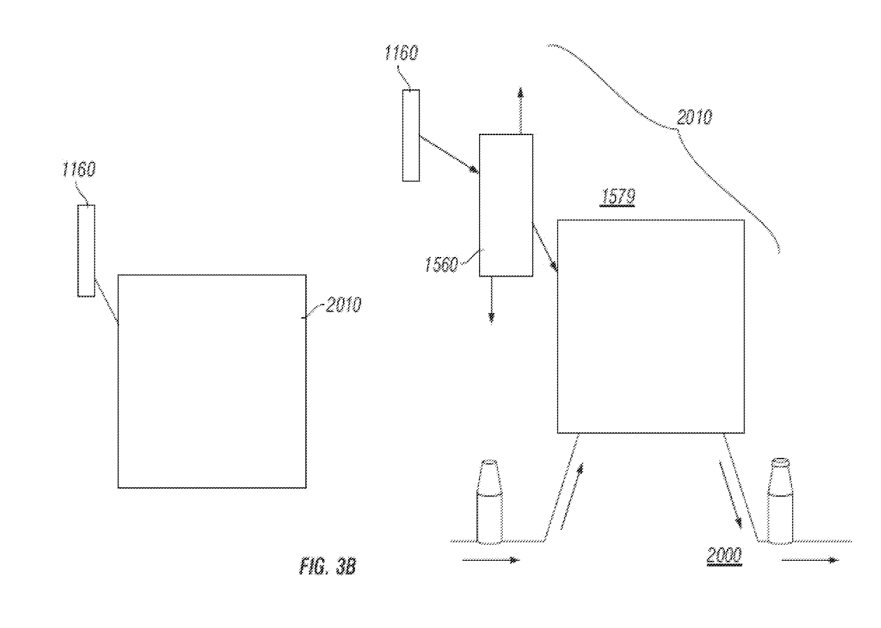

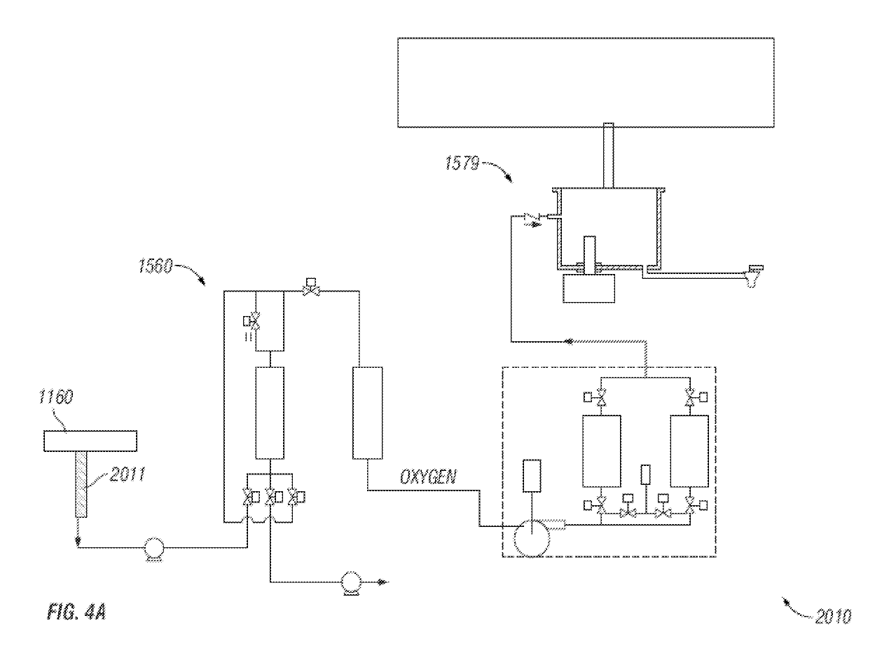

Referring now to FIGS. 3A and 3B, a functional block diagram of a fluid processing system 2010 and a macro view of the fluid processing system 2010 in accordance with embodiments disclosed herein, are shown. One major method of producing any oxygen gas stream from air involves passing a stream of clean, dry air through one bed of a pair of identical zeolite molecular sieves, which absorbs the nitrogen and delivers a gas stream that is 90% to 93% oxygen. Simultaneously, nitrogen gas is released from the other nitrogen-saturated zeolite bed, by reducing the chamber operating pressure and diverting part of the oxygen gas from the producer bed through it, in the reverse direction of flow. After a set cycle time the operation of the two beds is interchanged, thereby allowing for a continuous supply of gaseous oxygen to be pumped through a pipeline. This is known as pressure swing adsorption (hereinafter “PSA”). After separation, a purified stream may be used for additional purposes, such as in the processing of another fluid.

FIG. 3B illustrates using fluids captured by the FCM 1140, such as oxygen stored in the sealed chamber 1160. Although described as oxygen, it should be understood that any fluid captured by the FCM 1140 may be used within a fluid processing system 2010. The fluid processing system may include subsystems, such as a separation system 1560, as well as additional processing systems 1579 and 2000.

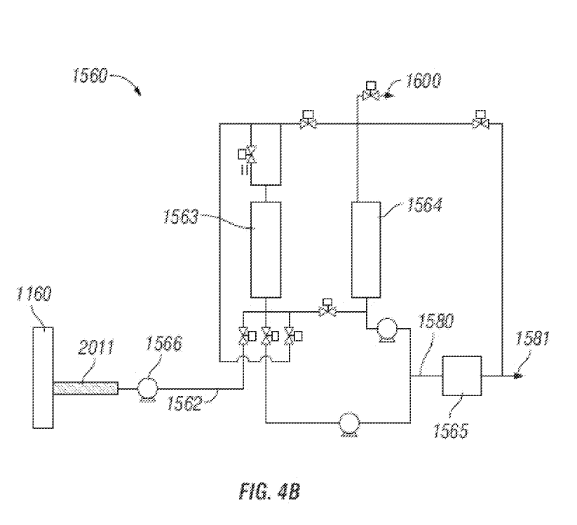

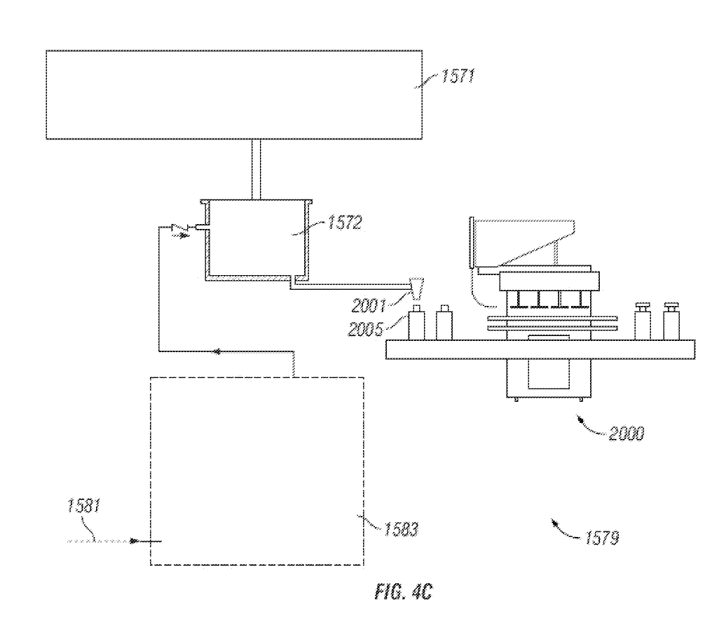

Referring now to FIGS. 4A, 4B, and 4C, multiple views of a fluid processing system 2000 in accordance with embodiments disclosed herein, are shown. As previously described, The SOLLEV 1000 may be used to capture atmospheric fluids, such as gases, liquids, and/or combinations thereof. As one example, the SOLLEV 1000 may be used to capture and recover atmospheric air. As another example, the SOLLEV may be used to capture and recover water vapor. The recovered fluid may subsequently be used for a variety of purposes, such as the (re)oxygenation of another fluid.

Thus, embodiments of the present disclosure may provide for a process to recover atmospheric fluid that may be used in the processing of another fluid. The recovered fluid may be separated into phases or components as may be desired. For example, atmospheric air may be separated into a substantially pure oxygen steam, such that the oxygen may be separated from a mixed gas that may be, for example, mainly composed of nitrogen gas and oxygen gas. Separation processes may include processes known in the art, such as U.S. Pat. No. 5,137,549 and U.S. Pat. No. 4,190,424, incorporated herein by reference. For example, the separation of oxygen from air may be by processes known to one of ordinary skill in the art, such as a well-known PSA.

Although the description that follows describes the separation of oxygen, and subsequent use of the separated and/or purified oxygen stream, embodiments of the disclosure are not meant to be limited by that description. For example, the captured fluid may be water vapor or carbon dioxide. These fluids may also be purified, studied, analyzed, used to process other fluids, etc.

Oxygen gas obtained by PSA may be used in various industrial fields wherein a large amount of oxygen is used continuously, for example, oxygen aeration for water treatment, bleaching, ozone generation and the like. Separation of oxygen, such as by PSA, has been generalized increasingly as a process for readily supplying oxygen at low costs.

Briefly, a conventional technique for producing an oxygen enriched gas by PSA has been predominantly employed a process which is designed to obtain an oxygen enriched gas in a high yield. The process usually includes flowing a mixed fluid stream that contains oxygen through one or more adsorbers. There may be repetitive respective steps for adsorption, recovery, desorption, pressurization, etc. in turn.

Thus, as shown by FIGS. 4A and 4B together, one embodiment of the disclosure may include the provision of a PSA system 1560 used to obtain a purified oxygen stream (in gas or liquid phase). The purified oxygen stream 1580 may have an oxygen concentration of 90% or more. A mixed gas 1562, such as atmospheric air mainly composed of nitrogen gas and oxygen gas, may be separated by the PSA system 1560. In one embodiment, the mixed gas 1562 may come from the sealed chamber 1160 of the FCM 1140. The PSA system 1560 may include, for example, a first adsorber 1563, as well as a second adsorber 1564. The adsorbers 1563 and 1564 may be packed with zeolite molecular sieve as the adsorbent, and a reservoir 1565 may used to accumulate the recovered oxygen stream 1580.

The reservoir 1565 may be connected to the outlet of each adsorber 1563 and 1564, and may be common to both adsorbers. The system 1560 may include introducing the mixed gas 1562 into one of the adsorbers 1563 or 1564 in order to adsorb nitrogen gas, and desorbing nitrogen gas previously adsorbed in another adsorber under reduced pressure. This process of the system 1560 may include, for example, introducing the mixed gas 1562 through the inlet of adsorber 1563 and selectively adsorbing nitrogen gas, while accumulating the resulting oxygen enriched gas in the reservoir 1565 through the outlet of adsorber 1563.

The process may also include desorbing nitrogen gas through the inlet of adsorber 1564, and also flowing a part of the oxygen enriched gas in the reservoir 1565 back to either of the adsorbers 1563 or 1564, whereby desorption of nitrogen gas may continue. The process may continue as would be known to one of ordinary skill in the art to produce a waste stream 1600, which may include any non-adsorbed nitrogen.

The zeolite molecular sieve (not shown) may selectively adsorb components, such as nitrogen gas, from the mixed fluid 1562. There may be one or more reservoirs 1565 connected to adsorbers 1563 and 1564, with associated tubing being provided therewith. The mixed fluid may be any gas or liquid. As one example, the mixed gas may include nitrogen gas and oxygen gas. The mixed fluid 1562 may be pressurized with a blower (i.e., pump, compressor, etc.) 1566, whereby the mixed fluid 1562 may then be introduced into one or more of the adsorbers 1563, 1564. Although the source of the mixed fluid 1566 is illustrated as the sealed chamber 1160, the mixed fluid may be fed from any source. In an embodiment, the mixed fluid 1562 may be fed directly into the system 1560 from the SOLLEV 1000.

In one example of a low pressure PSA process, the mixed gas may be pressurized between atmospheric pressure and less than 2 kg/cm² G, and the other adsorber (e.g., adsorber B) is desorbed and regenerated by reducing a pressure to 100 mmHg to 400 mmHg with a vacuum pump.

In another example of a high pressure PSA process, the mixed gas may be pressurized to 2 Kg/cm² G to 8 kg/cm² G and is introduced into adsorber 1563 to effect adsorption, and adsorber 1564 is desorbed and regenerated by releasing it in the atmosphere. However, the range of the pressure is not meant to be limited.

Nitrogen gas may be removed by adsorption so that the remainder product stream is an enriched oxygen gas stream 1580, which may be accumulated in a reservoir 1565. The adsorbers may be regenerated as would be known by one of skill in the art, such as reducing pressure and using vacuum pump.

Oxygen may be separated from atmospheric air by any number of conventional separation processes, such as by selective adsorption. In selective adsorption, atmospheric air may be pumped into one or more separators that have beds filled with a physical separation material. The physical separation material, such as 5A zeolite, permitted the less strongly adsorbed molecules such as oxygen and argon, to pass therethrough, but trapped or retained the more strongly adsorbed molecules of nitrogen, carbon dioxide, and water vapor.

Oxygen composes about 21% of atmospheric air whereas argon composes about 1%. When nitrogen, carbon dioxide, and other larger molecules are removed from atmospheric air leaving substantially only oxygen and argon, the percentages of both argon and oxygen in the separated gas increase about five fold. That is, even if the separator works perfectly, passing only oxygen and argon, the resultant product gas will be 4% to 5% argon and 95% to 96% oxygen. The purity of the resultant oxygen gas is theoretically limited by the argon content of atmospheric air to about 95.7% oxygen and 4.3% argon.

Once the separation process has reduced the mixed fluid stream 1562 to the desired component(s) stream, the component stream may be used for other beneficial purposes. For example, once oxygen is separated from the nitrogen and/or other impurities, the oxygen stream 1581 may be further used. In one embodiment, the purified oxygen may be used in the preparation and commercialization of fluids, such as water or sport enhancement drinks.

Referring now to FIGS. 4A and 4C, a view of an oxygenation process 1579 that may be part of fluid processing system 2010 in accordance with embodiments disclosed herein, is shown. The oxygenation of a fluid may occur by conventional methods, such as the oxygenation process of U.S. Pat. No. 5,006,352, fully incorporated by reference herein. Thus, the oxygen stream 1581 may be mixed with a stock fluid 1571. For example, the oxygen stream 1581 and the fluid 1571 may be cooled to between 0° C. and 5° C., followed by saturation with oxygen gas the same temperature in a saturating equipment or mixer 1572 at a pressure range of, for example, 0.3 to 0.4 MPa. During this process, flavoring agents, such as sugar, fructose, native flavors (e.g., apple, lemon, orange, plant extracts etc.) may be added.

Embodiments disclosed herein may provide for the oxygenation of a fluid, such as water. There may be an oxygen source 1581 connected with a delivery tank 1583, such that the tank 1583 may deliver oxygen into a mixer 1572. The oxygen source may include an oxygen generator (not shown). In one embodiment, the oxygen source may be the previously described PSA system 1560. The term water is intended to include, but without limitation, spring water, filtered water, water treated by the reverse osmosis, etc.

Oxygen enriched water may advantageously provide an enhanced taste and/or appeal. The dissolved oxygen content of the oxygenated water may be in the range of 5 mg/liter to mg/liter, depending on the source of the water and purification and processing techniques applied prior to bottling. The water may be supersaturated with oxygen by injecting oxygen into a mixer 1572 controlled at a pressure of, for example, 40-90 PSIG. Using this technique the dissolved oxygen level of the water may be increased to 25-125 mg/liter.

If immediately sealed, such as with sealing system 2000, the water may maintain the elevated dissolved oxygen level indefinitely. Oxygen concentration at saturation, as discussed in the foregoing, is proportional to the partial pressure of the oxygen in the contacting gas. The oxygen partial pressure in air at atmospheric pressure is 0.21 atmospheres, and the concentration of oxygen in water at saturation is about 9 mg. per liter at 70° F. (SATM conditions)

The present disclosure may provide for a new and improved system and method of operating the same for processing a fluid with another fluid. In an embodiment, the system an method include dispensing oxygen enriched water from a cooler/mixer 1572, the water having a dissolved oxygen content at an elevated level. The oxygen source preferably comprises an oxygen generator, such as a PSA system; however, the oxygen source can be of various other forms including stored oxygen such as bottled oxygen or the atmospheric capture mechanism.

Oxygen source 22 may be a small pressurized oxygen storage cylinder or an oxygen generation device which produces high purity oxygen from the PSA or the capture mechanism. In either case, the oxygen is delivered to the water tank 16 at a regulated pressure, such as 1-2 PSIG.

The water tank may be equipped with a refrigeration system to maintain the water dispensing temperature at or below 50 F. This device may consist of either a refrigerant compressor, condenser, and cooling coil or it may be a thermoelectric device.

Processes for oxygenating a liquid (e.g., preparing the liquid in solution with oxygen) are well known in the art. The liquid to be oxygenated may be water, for example, or any of a number of other liquids.

Currently, among the most effective methods and apparatuses for saturating a liquid with oxygen on an industrial scale are those disclosed in U.S. Pat. No. 5,766,490, which is expressly incorporated herein by reference, and in U.S. Pat. No. 6,120,008, also expressly incorporated herein by reference.

The process of the disclosure may provide for an oxygen-enriched liquid received from outlet 2001, at atmospheric pressure, which may have an oxygen level of at least about 160 mg/l. The fluid from the outlet 2001 may be poured into storage containers 2002, which may be subsequently closed or capped by system 2000. The storage containers may be glass, plastic, etc. One example of a container is polyester and even more specifically polyethylene terephthalate (PET) containers. Manufacturers and fillers, as well as consumers, have recognized that PET containers are lightweight, inexpensive, recyclable and manufacturable in large quantities.

Embodiments of the disclosure may also include methods of using apparatuses and systems previously described. Thus, there may be a method for oxygenating a fluid that includes launching an above-surface apparatus to a predetermined altitude greater than 20,000 feet above sea level, and while at the at the predetermined altitude, actuating an FCM sealingly configured to collect and store an atmospheric fluid. The method may also include returning the above-surface apparatus to an altitude less than 20,000 feet, followed by coupling the FCM, or any components of the FCM that are in fluid communication with the captured fluid, with an oxygenation process to mix the stored atmospheric fluid with the fluid.

The above-surface apparatus may include a launch device configured to overcome resistive forces in order to reach a layer of Earth's atmosphere that resides at a predetermined altitude greater than 20,000 feet. The FCM may be operatively connected to the launch device, and may be configured to acquire atmospheric gases at the predetermined altitude. The FCM may include a sealed chamber disposed in the FCM configured to store the captured atmospheric air, and a power source configured to provide power to the apparatus. The FCM may include a vacuum mechanism configured to move atmospheric air from the layer into a sealed chamber disposed in the FCM.

There may be a two-stage booster device disposed in the apparatus that includes a first stage and a second stage, wherein the first stage is configured as a non-propellant that launches the apparatus through a first predetermined layer of the atmosphere, and wherein the second stage comprises a propellant configured to maneuver the apparatus in a second predetermined layer of the atmosphere. In one embodiment, the layer may reside within the second predetermined layer.

The method may include other steps, such as separating the atmospheric air into at least one of a substantially pure nitrogen stream, a substantially pure oxygen stream, and combinations thereof. The separation may provide a substantially pure oxygen stream that includes subatomic particle properties in distinct quantities not present within at least one of synthetically formed oxygen, Earth-bound oxygen, and combinations thereof. After oxygenation, the method may include filling the fluid into at least one container, wherein the fluid comprises water having an oxygen content up to 25 g/ml. The oxygenation process may occur substantially near the Earth's surface, and may provide any oxygenated fluid having an oxygen content in the range of about 25 g/ml to 160 g/ml. The method may include taking a picture for visual analysis of the layer at the predetermined altitude.

Embodiments disclosed herein may provide for a method of operating a sub-orbital lower-level vehicle (SOLLEV) that includes the steps of launching the orbital apparatus to a layer of the Earth's atmosphere located at predetermined altitude greater than 20,000 feet above sea level. The SOLLEV may include a two-stage booster device further comprising a first stage and a second stage, wherein the first stage is configured with a non-combustible lighter-than-air material to launch the apparatus through a first predetermined layer of the atmosphere, and wherein the second stage comprises an ignitable material configured to maneuver the apparatus in a second predetermined layer of the atmosphere, and wherein the layer resides within the second predetermined layer.

Other steps may include performing an operation with the above-surface apparatus at the predetermined altitude, and returning the orbital apparatus to an altitude less than 20,000 feet. The method may be performed while over the continental United States. The method may include performing a number of operations. In an embodiment, the operation may include capturing atmospheric air. In another embodiment, the operation that comprises taking a picture at least a portion of the layer. Additional steps may include coupling at least a portion of the SOLLEV to an oxygenation process.

Embodiments disclosed herein may provide for one or more of the following advantages. Embodiments disclosed herein may provide for a less-expensive, cost-effective high atmosphere sub-orbital lower-level vehicle that may be used to perform various operations at altitudes not previously achieved. The vehicle may provide an inexpensive and reliable alternative to conventional launch systems. The launch costs will be significantly lower than for a rocket launch of a shuttle or satellite, including less than $1000. The operational costs are close to zero, while the entire system may be readily recovered as necessary, unlike a satellite which is either simply non-recoverable or requires an expensive space shuttle recovery.

Additional benefits and advantages include those obtained from oxygenated fluids. Experiments and research not previously achievable may now beneficially be performed. Thus, with the disclosure it is not possible to compare the same chemical element against those that may be substantially different at the subatomic level. How these differences may also affect humans differently when the chemical is consumed may also be beneficially studied. Embodiments disclosed herein may provide for an oxygenated fluid that may restore the human body after long-lasting and exhausting physical work.

The disclosure incorporates current executive branch strategy because it is exclusive to private industry. The disclosure also provides a cost effective above-surface apparatus that may be used to perform research and experimentation at the sub-orbital level. The above-surface apparatus is not tethered to the ground, and has substantially improved maneuverability. Research and analysis as it pertains to upper level atmospheric constituents, and their subsequent effect on humans, may now proceed in an economically viable manner.

Embodiments disclosed herein may provide for a restorative drink saturated with molecular oxygen. Lastly, the disclosure may beneficially provide for an above-surface apparatus that is safer, reliable, and more affordable.

While the present disclosure has been described with respect to a limited number of embodiments, those skilled in the art, having benefit of this disclosure, will appreciate that other embodiments may be devised which do not depart from the scope of the disclosure as described herein. Accordingly, the scope of the disclosure should be limited only by the attached claims.

What is claimed is:

1. A sub-orbital lower-level apparatus for collecting an atmospheric fluid, the apparatus comprising:
a main body;
a launch device coupled to the main body, wherein the launch device is configured to provide sufficient lift to the apparatus in order to overcome resistive forces to allow the apparatus to reach a layer of Earth's atmosphere that resides at a predetermined altitude greater than 20,000 feet above sea level; and
a fluid capture mechanism operatively connected to the main body and configured to acquire and store an atmospheric fluid.

2. The sub-orbital lower-level apparatus of claim 1, the apparatus further comprising:
a sealed chamber disposed in the fluid capture mechanism configured to store the captured atmospheric fluid, wherein the atmospheric fluid comprises air;
a power source configured to provide power to the apparatus; and
an actuator remotely operable to actuate the fluid capture mechanism at a predetermined time.

3. The sub-orbital lower-level apparatus of claim 2, the apparatus further comprising a camera configured to take an image while in the layer.

4. The sub-orbital lower-level apparatus of claim 2, the launch device further comprising: a two-stage booster device comprising a first stage and a second stage, wherein the first stage is configured with a lighter-than-air material to launch the apparatus through a first predetermined layer of the atmosphere, and wherein the second stage comprises a combustible material configured for ignition in order to maneuver the apparatus in a second predetermined layer of the atmosphere, and wherein the layer resides within the second predetermined layer.

5. The above-surface apparatus of claim 2, wherein the actuator is remotely operable via a timer mechanism configured to operate at a predetermined time.

6. The above-surface apparatus of claim 2, wherein the actuator is remotely operable via a wireless transmission initiated from an earthen surface.

7. The above-surface apparatus of claim 2, wherein the first stage further comprises a balloon and the lighter-than-air material comprises a non-combustible gas, and wherein the second stage further comprises at least one thruster configured to exhaust gas that results from ignition of the combustible material.

8. The above-surface apparatus of claim 1, the launch device further comprising:
a two-stage booster device further comprising a first stage and a second stage, wherein the first stage is configured with a non-propellant to launch the apparatus through a first predetermined layer of the atmosphere, and wherein the second stage comprises a propellant configured to maneuver the apparatus in a second predetermined layer of the atmosphere, and wherein the layer resides within the second predetermined layer.

9. The above-surface apparatus of claim 8, wherein the layer resides at a predetermined altitude greater than 40,000 feet.

10. The above-surface apparatus of claim 8, the apparatus further comprising a camera configured to take an image while the launch device is maneuvering in the layer.

11. The above-surface apparatus of claim 10, wherein the image comprises a second image disposed in the viewing range of the camera.

12. The above-surface apparatus of claim 1, wherein the layer resides at an altitude in the range from about 40,000 feet to an altitude substantially adjacent the Earth's atmosphere that comprises a natural vacuum.

13. The above-surface apparatus of claim 1, wherein the layer resides at a predetermined altitude greater than 40,000 feet.

14. A method for oxygenating a fluid, the method comprising:
launching a sub-orbital lower-level apparatus to a predetermined altitude greater than 20,000 feet above sea level;
at the predetermined altitude, actuating a fluid capture mechanism that is sealingly configured to collect and store an atmospheric fluid;
returning the a sub-orbital lower-level apparatus to an altitude less than 20,000 feet;
introducing the atmospheric fluid to an oxygenation process to mix the captured atmospheric fluid with another fluid.

15. The method of claim 14, wherein the a sub-orbital lower-level apparatus comprises:
a launch device configured to overcome resistive forces in order to reach a layer of Earth's atmosphere that resides at a predetermined altitude greater than 20,000 feet, wherein the fluid capture mechanism is operatively connected to the launch device and configured to acquire atmospheric gases at the predetermined altitude;
at least one sealed chamber disposed in the fluid capture mechanism configured to store the captured atmospheric fluid; and
a power source configured to provide power to the apparatus.

16. The method of claim 15, wherein the a sub-orbital lower-level apparatus further comprises:
a two-stage booster device further comprising a first stage and a second stage, wherein the first stage is configured with a non-propellant material that launches the apparatus through a first predetermined layer of the atmosphere, and wherein the second stage comprises a propellant material configured to ignite in order to maneuver the apparatus in a second predetermined layer of the atmosphere, and wherein the layer resides within the second predetermined layer.

17. The method of claim 14, the method further comprising:
separating the atmospheric fluid into at least one purified stream, wherein the atmospheric fluid comprises air, and the at least one purified streams is one of a substantially pure nitrogen stream, a substantially pure oxygen stream, and combinations thereof.

18. The method of claim 17, wherein the substantially pure oxygen stream comprises subatomic particles in distinct quantities not present within at least one of synthetically formed oxygen, Earth-bound oxygen, and combinations thereof.

19. The method of claim 14, the method further comprising:
after oxygenation, filling the oxygenated fluid into at least one container, wherein the oxygenated fluid comprises water having an oxygen content of about 20 mg/L.

20. The method of claim 14, wherein the fluid capture mechanism is coupled with a vacuum mechanism configured to move atmospheric fluid from the layer into a sealed chamber disposed in the fluid capture mechanism.

21. The method of claim 14, the method further comprising taking a picture for visual analysis of the layer at the predetermined altitude.

22. The method of claim 14, wherein the oxygenation process is located substantially near the Earth's surface.

23. A method of operating a sub-orbital lower-level apparatus, the method comprising:
launching the sub-orbital lower-level apparatus to a layer of the Earth's atmosphere located at predetermined altitude greater than 20,000 feet above sea level, the sub-orbital lower-level apparatus comprising:
a two-stage booster device further comprising a first stage and a second stage, wherein the first stage is configured with a non-combustible lighter-than-air material to provide enough lift to launch the apparatus through a first predetermined layer of the atmosphere, and wherein the second stage comprises a material configured ignite, whereby ignition of the material produces a resultant force to maneuver the apparatus in a second predetermined layer of the atmosphere, and wherein the layer resides within the second predetermined layer performing an operation with the sub-orbital lower-level apparatus at the predetermined altitude; and
returning the sub-orbital lower-level apparatus to an altitude less than 20,000 feet.

24. The method of claim 23, the method further comprising performing the operation while over the continental United States.

25. The method of claim 23, wherein the performing the operation comprises capturing atmospheric air.

26. The method of claim 25, the method further comprising performing a second operation that comprises taking a picture at least a portion of the layer.

27. The method of claim 23, the method further comprising the step of coupling at least a portion of the sub-orbital lower-level apparatus to an oxygenation process.

28. The method of claim 23, wherein the performing the operation comprises taking a picture of at least a portion of the layer.
